(19) United States
(12) Patent Application Publication
(10) Pub. No.: US 2024/0379215 A1
(43) Pub. Date: Nov. 14, 2024

(54) VR TRAINING AND TREATMENT SYSTEM AND METHOD THEREOF

(71) Applicant: Kryn McClain, Reading, PA (US)

(72) Inventor: Kryn McClain, Reading, PA (US)

(21) Appl. No.: 18/196,140

(22) Filed: May 11, 2023

(51) Int. Cl. G16H 20/70; G02B 27/01; G06F 3/01; G16H 10/20

(52) U.S. Cl. CPC G16H 20/70; G02B 27/017; G06F 3/011; G16H 10/20

(57) ABSTRACT

Disclosed is an information processing apparatus that includes processing circuitry that is coupled to a Virtual Reality (VR) headset. The processing circuitry is configured to (i) display, by way of the VR headset, a set of predefined training modules within a VR environment such that upon display of the set of training modules, the first user selects at least one training module of the set of training modules and (ii) generate, based on the selected at least one training module of the set of training modules, a module specific VR environment for the first user such that the module specific VR environment is generated by way of a pre-recorded live footage that is associated with the selected at least one training module of the set of training modules.

### BACKGROUND

The present disclosure relates generally to Virtual Reality (VR) system and method, and more particularly, to an apparatus, a system and a method for training people with autism, or any type of neurodiversity by way of a VR headset.

Autism, also known as Autism Spectrum Disorder (ASD), refers to a neurodevelopmental disorder in which affected individual commonly show difficulties engaging in social interactions and communication and repetitive behaviors. Although there is no known cure, today, autism therapy ranges across specialties including neurology, psychiatry, physical therapy, occupational therapy, behavioral therapy, and speech language pathology. Such therapeutic methods may help individuals with ASD to gain social and communication skills. This can also be used for individuals who suffer from other types of neurodiversity due to stroke, head injuries, or other neurological conditions. Additionally, these modules can be adapted to be used for training individuals who work with or interact with neurodiverse conditions.

Virtual Reality (VR) is a coordinated experience generated by and housed in computer processors and memory. VR systems are often used to create simulations so that a user may learn, or train while being exposed to little or no physical risk. Generally, VR systems utilizes animation for training individuals having neurodiversity with the help of a game-based approach, eye movement tracking and the feedback related to the tracked movements, and the like. However, such approaches lack in providing real life experience-based training to the individuals having ASD or other neurologically diverse issues, that can improve psychosocial functioning and quality of life by raising the individual's level of independence in activities of daily living.

Thus, there is a need for a technical solution that overcomes the aforementioned problems of conventional systems and methods to train an individual having neurodiverse issues.

### SUMMARY

In an aspect of the present disclosure, an information processing apparatus is disclosed. The information processing apparatus includes processing circuitry that is coupled to a Virtual Reality (VR) headset. The processing circuitry is configured to (i) display, by way of the VR headset, a set of predefined training modules within a VR environment such that upon display of the set of training modules, the first user selects at least one training module of the set of training modules and (ii) generate, based on the selected at least one training module of the set of training modules, a module specific VR environment for the first user such that the module specific VR environment is generated by way of a pre-recorded live footage that is associated with the selected at least one training module of the set of training modules. The training modules are for any users who have neurodiverse conditions, but can also be adapted to include training to individuals who work with neurodiverse individuals.

In some embodiments of the present disclosure, each training module of the set of training modules includes one or more stepwise instructions to perform one or more tasks such that each task is adapted to enhance a skill of the first user.

In some embodiments of the present disclosure, each task of the one or more tasks includes a plurality of actions to be performed by the user, wherein the plurality of actions includes (i) an action performed in a correct order and (ii) an action performed in an incorrect order.

In some embodiments of the present disclosure, the processing circuitry is further configured to identify one of the plurality of actions performed by the user based on one or more sensed signals received from a plurality of sensors. The one or more sensed signals represents one or more parameters associated with the first user.

In some embodiments of the present disclosure, the processing circuitry is further configured to capture, by way of one or more imaging devices, a real time video footage of the first user while the first user performs a task of the one or more tasks and display, by way of a second user device associated with a second user, the real time video footage.

In some embodiments of the present disclosure, the processing circuitry is further configured to enable the second user, by way of the second user device, to (i) provide one or more instructions to the first user, (ii) monitor the first user, or a combination thereof.

In some embodiments of the present disclosure, the processing circuitry is further configured to enable, by way of the second user device, the second user to provide real-time commands to the first user within the module specific VR environment.

In some embodiments of the present disclosure, the processing circuitry is further configured to generate a score. To generate the score, the processing circuitry is configured to track an answer provided by the first user to each question of one or more questions associated with the selected at least one training module of the set of training modules.

In another aspect of the present disclosure, a system is disclosed. The system includes a Virtual Reality (VR) headset that is adapted to be worn by a first user; processing circuitry that is coupled to the VR headset, and configured to: display, by way of the VR headset, a set of predefined training modules within a VR environment such that upon display of the set of training modules, the first user selects at least one training module of the set of training modules and generate, based on the selected at least one training module of the set of training modules, a module specific VR environment for the first user such that the module specific VR environment is generated by way of a pre-recorded live footage that is associated with the selected at least one training module of the set of training modules.

In yet another aspect of the present disclosure, a computer-implemented method is disclosed. The method when executed on processing circuitry causes the processing circuitry to perform operations including displaying, by way of a VR headset, a set of predefined training modules within a VR environment such that upon display of the set of training modules, a first user selects at least one training module of the set of training modules and generating, based on the selected at least one training module of the set of training modules, a module specific VR environment for the first user such that the module specific VR environment is generated by way of a pre-recorded live footage that is associated with the selected at least one training module of the set of training modules.

In some embodiments of the present disclosure, the method further includes sensing by way of a plurality of sensors, one or more signals representing parameters associated with the first user.

In some embodiments of the present disclosure, the method further includes identifying, by way of the processing circuitry, at least one of the plurality of actions performed by the first user based on one or more sensed signals received from the plurality of sensors.

In some embodiments of the present disclosure, the method further includes capturing, by way of one or more imaging devices, a real time video footage of the first user while the first user performs each task of the one or more tasks and displaying, by way of the second user device, the real time video footage.

In some embodiments of the present disclosure, the method further includes generating, by way of the processing circuitry, a score by tracking an answer provided by the first user to each question of one or more questions associated with the selected at least one training module of the set of training modules.

### BRIEF DESCRIPTION OF THE DRAWINGS

The following detailed description of the preferred embodiments of the present disclosure will be better understood when read in conjunction with the appended drawings. The present disclosure is illustrated by way of example, and not limited by the accompanying figures, in which like references indicate similar elements.

FIG. 1 is a block diagram that illustrates a system to train a user having Autism Spectrum Disorder (ASD) by way of Virtual Reality (VR), in accordance with an embodiment of the present disclosure;

FIG. 2 is a block diagram that illustrates an information processing apparatus of FIG. 1, in accordance with an exemplary embodiment of the present disclosure; and

FIG. 3 illustrates a flowchart of a method for training a user having ASD by way of VR, in accordance with an embodiment of the present disclosure.

### DETAILED DESCRIPTION

The detailed description of the appended drawings is intended as a description of the currently preferred embodiments of the present disclosure and is not intended to represent the only form in which the present disclosure may be practiced. It is to be understood that the same or equivalent functions may be accomplished by different embodiments that are intended to be encompassed within the spirit and scope of the present disclosure.

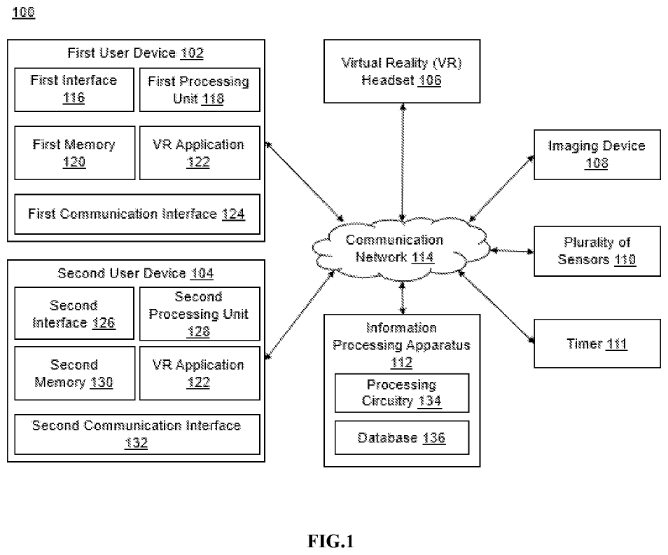

FIG. 1 is a block diagram that illustrates a system 100 to train a user having Autism Spectrum Disorder (ASD) or any type of neurodiverse condition, in accordance with an embodiment of the present disclosure. The system 100 may facilitate to teach not only skills but muscle memory and repetition to the user (hereinafter interchangeably referred to as “the first user”) having ASD. Specifically, the system 100 may be configured to utilize Virtual Reality (VR) technology where content may be shot in real-world, edited, and presented to allow the first user to input answers directly into the system 100. The system 100 may include a first user device 102, a second user device 104, a VR headset 106, an imaging device 108, a plurality of sensors 110, a timer 111, and an information processing apparatus 112. The first user device 102, the second user device 104, the VR headset 106, the imaging device 108, the plurality of sensors 110, the timer 111, and the information processing apparatus 112 may be configured to communicate with each other and other entities within the system 100 by way of a communication network 114 or through separate communication networks established therebetween.

The communication network 114 may include suitable logic, circuitry, and interfaces that may be configured to provide a plurality of network ports and a plurality of communication channels for transmission and reception of data related to operations of various entities in the system 100. Each network port may correspond to a virtual address (or a physical machine address) for transmission and reception of the communication data. For example, the virtual address may be an Internet Protocol Version 4 (IPV4) (or an IPV6 address) and the physical address may be a Media Access Control (MAC) address. The communication network 114 may be associated with an application layer for implementation of communication protocols based on one or more communication requests from the first user device 102, the second user device 104, the VR headset 106, the imaging device 108, the plurality of sensors 110, and the information processing apparatus 112. The communication data may be transmitted or received, via the communication protocols. Examples of the communication protocols may include, but are not limited to, Hypertext Transfer Protocol (HTTP), File Transfer Protocol (FTP), Simple Mail Transfer Protocol (SMTP), Domain Network System (DNS) protocol, Common Management Interface Protocol (CMIP), Transmission Control Protocol and Internet Protocol (TCP/IP), User Datagram Protocol (UDP), Long Term Evolution (LTE) communication protocols, or any combination thereof.

In one embodiment, the communication data may be transmitted or received via at least one communication channel of a plurality of communication channels in the communication network 114. The communication channels may include, but are not limited to, a wireless channel, a wired channel, a combination of wireless and wired channel thereof. The wireless or wired channel may be associated with a data standard which may be defined by one of a Local Area Network (LAN), a Personal Area Network (PAN), a Wireless Local Area Network (WLAN), a Wireless Sensor Network (WSN), Wireless Area Network (WAN), Wireless Wide Area Network (WWAN), a Metropolitan Area Network (MAN), a Satellite Network, the Internet, a Fiber Optic Network, a Coaxial Cable Network, an Infrared (IR) network, a Radio Frequency (RF) network, and a combination thereof. Embodiments of the present invention are intended to include or otherwise cover any type of communication channel, including known, related art, and/or later developed technologies.

The first user device 102 may be adapted to facilitate a user (e.g., the first user) to input data, receive data, and/or transmit data within the system 100. In some embodiments of the present disclosure, the first user device 102 may include, but is not limited to, a desktop, a notebook, a laptop, a handheld computer, a touch sensitive device, a computing device, a smart phone, a smart watch, and the like. It will be apparent to a person of ordinary skill in the art that the first user device 102 may include any device/apparatus that is capable of manipulation by the first user. Although FIG. 1 illustrates that the system 100 includes a single first user device (i.e., the first user device 102), it will be apparent to a person skilled in the art that the scope of the present disclosure is not limited to it. In various other aspects, the system 100 may include multiple first user devices without deviating from the scope of the present disclosure. In such a scenario, each first user device is configured to perform one or more operations in a manner similar to the operations of the first user device 102 as described herein. As illustrated, the first user device 102 includes a first interface 116, a first processing unit 118, and a first memory 120.

The first interface 116 may include an input interface for receiving inputs from the first user. Examples of the input interface may include, but are not limited to, a touch interface, a mouse, a keyboard, a motion recognition unit, a gesture recognition unit, a voice recognition unit, or the like. Embodiments of the present disclosure are intended to include or otherwise cover any type of the input interface including known, related art, and/or later developed technologies. The first interface 116 may further include an output interface for displaying (or presenting) an output to the customer. Examples of the output interface may include, but are not limited to, a display device, a printer, a projection device, and/or a speaker. Examples of the first interface 116 may include, but are not limited to, a digital display, an analog display, a touch screen display, a graphical customer interface, a website, a webpage, a keyboard, a mouse, a light pen, an appearance of a desktop, and/or illuminated characters.

The first processing unit 118 may include suitable logic, instructions, circuitry, and/or interfaces for executing various operations, such as one or more operations associated with the first user device 102. In some embodiments of the present disclosure, the first processing unit 118 may be configured to control the one or more operations executed by the first user device 102 in response to an input received at the first user device 102 from the first user. Examples of the first processing unit 118 may include, but are not limited to, an Application-Specific Integrated Circuit (ASIC) processor, a Reduced Instruction Set Computing (RISC) processor, a Complex Instruction Set Computing (CISC) processor, a Field-Programmable Gate Array (FPGA), a Programmable Logic Control unit (PLC), and the like. Embodiments of the present disclosure are intended to include or otherwise cover any type of the first processing unit 118 including known, related art, and/or later developed technologies.

The first memory 120 may be configured to store logic, instructions, circuitry, interfaces, and/or codes of the first processing unit 118, data associated with the first user device 102, and data associated with the system 100. Examples of the first memory 120 may include, but are not limited to, a Read Only Memory (ROM), a Random Access Memory (RAM), a Flash Memory (FM), a Removable Storage Drive (RSD), a Hard Disk Drive (HDD), a Solid-State Memory (SSM), a Magnetic Storage Drive (MSD), a Programmable Read Only Memory (PROM), an Erasable PROM (EPROM), and/or an Electrically EPROM (EEPROM). Embodiments of the present disclosure are intended to include or otherwise cover any type of the first memory 120 including known, related art, and/or later developed technologies.

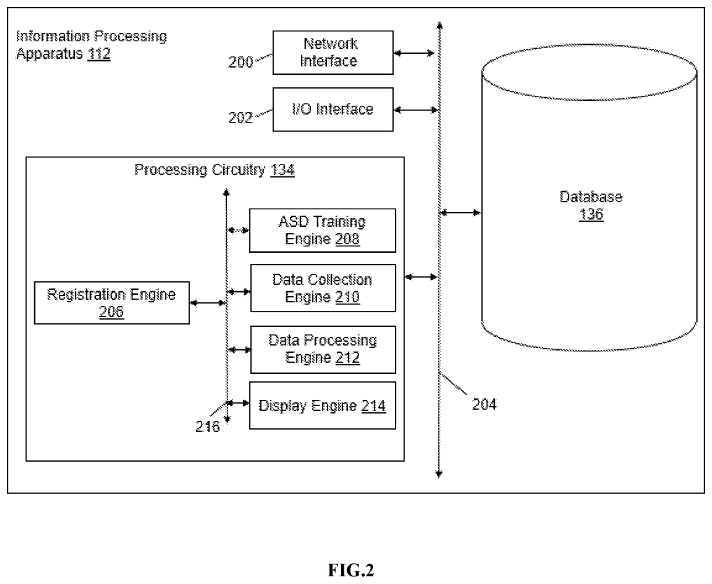

In some embodiments of the present disclosure, the first user device 102 may further include one or more computer executable applications configured to be executed by the first processing unit 118. The one or more computer executable applications may include suitable logic, instructions, and/or codes for executing various operations associated with the system 100. The one or more computer executable applications may be stored in the first memory 120. Examples of the one or more computer executable applications may include, but are not limited to, an audio application, a video application, a social media application, a navigation application, and the like. Preferably, the one or more computer executable applications may include a VR application 122. Specifically, one or more operations associated with the VR application 122 may be controlled by the information processing apparatus 112 that will be explained in detail in FIG. 2.

The first user device 102 may further include a first communication interface 124. The first communication interface 124 may be configured to enable the first user device 102 to communicate with the information processing apparatus 112 and other components of the system 100 over the communication network 114. Examples of the first communication interface 124 may include, but are not limited to, a modem, a network interface such as an Ethernet Card, a communication port, and/or a Personal Computer Memory Card International Association (PCMCIA) slot and card, an antenna, a Radio Frequency (RF) transceiver, one or more amplifiers, a tuner, one or more oscillators, a digital signal processor, a Coder Decoder (CODEC) Chipset, a Subscriber Identity Module (SIM) card, and a local buffer circuit. It will be apparent to a person of ordinary skill in the art that the first communication interface 124 may include any device and/or apparatus capable of providing wireless and/or wired communications between the first user device 102 and the information processing apparatus 112.

The second user device 104 may be adapted to facilitate a user (e.g., a second user) to input data, receive data, and/or transmit data within the system 100. In some embodiments of the present disclosure, the second user may be, but is not limited to, a professional (including clinicians who provide services to users with autism and who may adopt practices based on data generated by the system 100), a researcher (who may use the data generated by the system 100 to inform new studies), policy makers (who are essential in effecting change), a family member and/or caretaker of the first user (i.e., a person having autism), and the like. Embodiments of the present disclosure are intended to include and/or otherwise cover any type of the second user, without deviating from the scope of the present disclosure. In some embodiments of the present disclosure, the second user device 104 may include, but is not limited to, a desktop, a notebook, a laptop, a handheld computer, a touch sensitive device, a computing device, a smart phone, a smart watch, and the like. It will be apparent to a person of ordinary skill in the art that the second user device 104 may include any device/apparatus that is capable of manipulation by the second user. Although FIG. 1 illustrates that the system 100 includes a single second user device (i.e., the second user device 104), it will be apparent to a person skilled in the art that the scope of the present disclosure is not limited to it. In various other aspects, the system 100 may include multiple second user devices without deviating from the scope of the present disclosure. In such a scenario, each second user device is configured to perform one or more operations in a manner similar to the operations of the second user device 104 as described herein. As illustrated, the second user device 104 includes a second interface 126, a second processing unit 128, and a second memory 130.

The second interface 126 may include an input interface for receiving inputs from the second user. Examples of the input interface may include, but are not limited to, a touch interface, a mouse, a keyboard, a motion recognition unit, a gesture recognition unit, a voice recognition unit, or the like. Embodiments of the present disclosure are intended to include or otherwise cover any type of the input interface including known, related art, and/or later developed technologies. The second interface 126 may further include an output interface for displaying (or presenting) an output to the customer. Examples of the output interface may include, but are not limited to, a display device, a printer, a projection device, and/or a speaker. Examples of the second interface 126 may include, but are not limited to, a digital display, an analog display, a touch screen display, a graphical customer interface, a website, a webpage, a keyboard, a mouse, a light pen, an appearance of a desktop, and/or illuminated characters.

The second processing unit 128 may include suitable logic, instructions, circuitry, and/or interfaces for executing various operations, such as one or more operations associated with the second user device 104. In some embodiments of the present disclosure, the second processing unit 128 may be configured to control the one or more operations executed by the second user device 104 in response to an input received at the second user device 104 from the second user. Examples of the second processing unit 128 may include, but are not limited to, an Application Specific Integrated Circuit (ASIC) processor, a Reduced Instruction Set Computing (RISC) Processor, a Complex Instruction Set Computing (CISC) Processor, a Field Programmable Gate Array (FPGA), a Programmable Logic Control unit (PLC), and the like. Embodiments of the present disclosure are intended to include or otherwise cover any type of the second processing unit 128 including known, related art, and/or later developed technologies.

The second memory 130 may be configured to store logic, instructions, circuitry, interfaces, and/or codes of the second processing unit 128, data associated with the second user device 104, and data associated with the system 100. Examples of the second memory 130 may include, but are not limited to, a ROM, a RAM, a Flash Memory, a Removable Storage Drive, a HDD, a Solid State Memory, a Magnetic Storage Drive, a PROM, an EPROM, and/or an EEPROM. Embodiments of the present disclosure are intended to include or otherwise cover any type of the second memory 130 including known, related art, and/or later developed technologies.

In some embodiments of the present disclosure, the second user device 104 may further include one or more computer executable applications configured to be executed by the second processing unit 128. The one or more computer executable applications may include suitable logic, instructions, and/or codes for executing various operations associated with the system 100. The one or more computer executable applications may be stored in the second memory 130. Examples of the one or more computer executable applications may include, but are not limited to, an audio application, a video application, a social media application, a navigation application, and the like. Preferably, the one or more computer executable applications may include the VR application 122 (as discussed above).

The second user device 104 may further include a second communication interface 132. The second communication interface 132 may be configured to enable the second user device 104 to communicate with the information processing apparatus 112 and other components of the system 100 over the communication network 114. Examples of the second communication interface 132 may include, but are not limited to, a modem, a network interface such as an Ethernet Card, a communication port, and/or a PCMCIA slot and card, an antenna, a RF transceiver, one or more amplifiers, a tuner, one or more oscillators, a digital signal processor, a CODEC chipset, a SIM card, and a local buffer circuit. It will be apparent to a person of ordinary skill in the art that the second communication interface 132 may include any device and/or apparatus capable of providing wireless and/or wired communications between the second user device 104 and the information processing apparatus 112.

The imaging device 108 may be installed in a vicinity where the first user is to be positioned to experience the module specific VR environment. Specifically, the imaging device 108 may be installed in a way such that a field of view of the imaging device 108 substantially covers an area where the first user is to be positioned to experience the module specific VR environment. In some embodiments of the present disclosure, the imaging device 108 may be configured to capture a real-time footage of the first user while performing a task of a plurality of tasks of associated with the set of training modules. In some embodiments of the present disclosure, the imaging device 108 may be, but not limited to, a digital camera, an analog camera, a smartphone camera, and the like. Embodiments of the present disclosure are intended to include and/or otherwise cover any type of the imaging device 108, including known, related, and later developed technologies, without deviating from the scope of the present disclosure.

The plurality of sensors 110 may be installed in the vicinity where the first user is to be positioned to experience the module specific VR environment. Specifically, the plurality of sensors 110 may be installed in a way such that a field of view of the plurality of sensors 110 substantially covers an area where the first user is to be positioned to experience the module specific VR environment. In some embodiments of the preset disclosure, the plurality of sensors 110 may be configured to sense signals that represents one or more parameters associated with the first user. For example, the parameters may include, but is not limited to, a movement executed by the first user, gestures of the first user, and the like. Embodiments of the present disclosure are intended to include and/or otherwise cover any type of the parameters associated with the first user that may facilitate to efficiently monitor the first user while the first user performs a task of the one or more tasks associated with the set of training modules, without deviating from the scope of the present disclosure. In some embodiments of the present disclosure, the plurality of sensors 110 may be configured to sense signals that represents an action performed by the first user. In some embodiments of the present disclosure, the plurality of sensors 110 may include, but is not limited to, a proximity sensor, an Ultrasonic sensor, and the like. Embodiments of the present disclosure are intended to include and/or otherwise cover any type of the plurality of sensors 110, including known, related, and later developed sensors, without deviating from the scope of the present disclosure.

The timer 111 may be a timing clock that may be configured to record a timing data associated with the first user while performs at least one training module of the set of training modules. In some embodiments of the present disclosure, the timer 111 may be, but not limited to, a digital clock, an analog clock, and the like. Embodiments of the present disclosure are intended to include and/or otherwise cover any type of the timer 111, including known, related, and later developed technologies, without deviating from the scope of the present disclosure.

The information processing apparatus 112 may be a network of computers, a framework, or a combination thereof, that may provide a generalized approach to create a server implementation. In some embodiments of the present disclosure, the information processing apparatus 112 may be a server. Examples of the information processing apparatus 112 may include, but are not limited to, personal computers, laptops, mini-computers, mainframe computers, any non-transient and tangible machine that can execute a machine-readable code, cloud-based servers, distributed server networks, or a network of computer systems. The information processing apparatus 112 may be realized through various web-based technologies such as, but not limited to, a Java web-framework, a.NET framework, a personal home page (PHP) framework, or any other web-application framework. The information processing apparatus 112 may include one or more processing circuitries of which processing circuitry 134 is shown and a non-transitory computer-readable storage medium 136.

The processing circuitry 134 may be configured to execute various operations associated with the system 100. The processing circuitry 134 may be configured to host and enable the VR application 122 running on (and/or installed on) the first and second user devices 102 and 104 to execute the one or more operations associated with the system 100 by communicating one or more commands and/or instructions over the communication network 114. Examples of the processing circuitry 134 may include, but are not limited to, an ASIC processor, a RISC processor, a CISC processor, a FPGA, and the like. Embodiments of the present disclosure are intended to include and/or otherwise cover any type of the processing circuitry 134 including known, related art, and/or later developed technologies.

The non-transitory computer-readable storage medium 136 (hereinafter interchangeably referred to and designated as “the database 136”) may be configured to store the logic, instructions, circuitry, interfaces, and/or codes of the processing circuitry 134 for executing various operations. The database 136 may be further configured to store therein, data associated with the users registered with the system 100, and the like. It will be apparent to a person having ordinary skill in the art that the database 136 may be configured to store various types of data associated with the system 100, without deviating from the scope of the present disclosure. Examples of the database 136 may include but are not limited to, a Relational database, a NoSQL database, a Cloud database, an Object-oriented database, and the like. Further, the database 136 may include associated memories that may include, but is not limited to, a ROM, a RAM, a flash memory, a removable storage drive, a HDD, a solid-state memory, a magnetic storage drive, a PROM, an EPROM, and/or an EEPROM. Embodiments of the present disclosure are intended to include or otherwise cover any type of the database 136 including known, related art, and/or later developed technologies. In some embodiments of the present disclosure, a set of centralized or distributed network of peripheral memory devices may be interfaced with the server 104, as an example, on a cloud server.

In operation, when the system 100 identifies that the first user wearing the VR headset 106 has activated the VR headset 106 by way of the first user device 102 such that. The system 100, by way of the VR headset 106 may display a set of predefined training modules within a VR environment. Further, the system 100 may guide the first user by way of one or more real-time commands to select at least one training module of the set of training modules. In some embodiments of the present disclosure, each training module of the set of training modules may include one or more stepwise instructions to perform one or more tasks such that each task is adapted to enhance a skill of the first user. Further, each task of the one or more tasks may include a plurality of actions to be performed by the user, wherein the plurality of actions may include (i) an action performed in a correct order and (ii) an action performed in an incorrect order. Furthermore, the system 100 may sense by way of the plurality of sensors 100, one or more signals representing parameters associated with the first user. Specifically, the system 100 may identify at least one of the plurality of actions performed by the first user based on one or more sensed signals received from the plurality of sensors. In some embodiments of the present disclosure, each training module of the set of training modules may include one or more one or more questions related to the training module (and/or a therapy program) that the first user is participating in. The system 100 may be configured to track an answer provided by the first user to each question of the one or more questions. Specifically, the system 100 is configured to track a progress of a performance of the first user by collecting data (i.e., the answers to the one or more questions). Further, the system 100 may provide a score at the end of the training module that represents a number of correct answers out of the total number of questions asked from the first user. For example, when the first user answers 14 out of 20 questions correctly, the system 100 displays a score of 14/20 to indicate a level of proficiency in the training module being covered. The system 100 may be configured to utilize such tracking and feedback mechanism to monitor a progress of the first user over time and adjust a program accordingly to ensure optimal outcomes.

Further, the system 100 may record a timing data associated with the first user while the first user performs at least one training module of the set of training modules. Next, the system 100 may generate, based on the selected at least one training module of the set of training modules, a module specific VR environment for the first user such that the module specific VR environment is generated by way of a pre-recorded live footage that is associated with the selected at least one training module of the set of training modules. Further, the system 100 may guide the first user by way of one or more real-time commands to perform a task of the one or more tasks. Further, the system 100 may identify an action of the plurality of actions performed by the first user based on one or more sensed signals received from the plurality of sensors while the first user performs the task of the one or more tasks. Furthermore, the system 100 may capture, by way of one or more imaging devices, a real time video footage of the first user while the first user performs the task of the one or more tasks. Next, the system 100 may display, by way of the second user device associated with the second user, the real time video footage such that the second user can monitor the first user while in the module specific VR environment.

FIG. 2 is a block diagram that illustrates the information processing apparatus 112 of FIG. 1, in accordance with an embodiment of the present disclosure. As discussed, the information processing apparatus 112 includes the processing circuitry 134 and the database 136. Further, the information processing apparatus 112 may include a network interface 200 and an input/output (I/O) interface 202. The processing circuitry 134, the database 136, the network interface 200, and the I/O interface 202 may communicate with each other by way of a first communication bus 204. In some embodiments of the present disclosure, the processing circuitry 134 may include a registration engine 206, an ASD training engine 208, a data collection engine 210, a data processing engine 212, and a display engine 214. The registration engine 206, the ASD training engine 208, the data collection engine 210, the data processing engine 212, and the display engine 214 may communicate with each other by way of a second communication bus 216. It will be apparent to a person having ordinary skill in the art that the information processing apparatus 112 is for illustrative purposes and not limited to any specific combination of hardware circuitry and/or software.

The network interface 200 may include suitable logic, circuitry, and interfaces that may be configured to establish and enable a communication between the information processing apparatus 112 and different components of the system 100 (e.g., the first and second user devices 102 and 104), via the communication network 114. The network interface 200 may be implemented by use of various known technologies to support wired or wireless communication of the information processing apparatus 112 with the communication network 114. The network interface 200 may include, but is not limited to, an antenna, a RF transceiver, one or more amplifiers, a tuner, one or more oscillators, a digital signal processor, a CODEC chipset, a SIM card, and a local buffer circuit.

The I/O interface 202 may include suitable logic, circuitry, interfaces, and/or code that may be configured to receive inputs and transmit server outputs (i.e., one or more outputs generated by the information processing apparatus 112) via a plurality of data ports in the information processing apparatus 112. The I/O interface 202 may include various input and output data ports for different I/O devices. Examples of such I/O devices may include, but are not limited to, a touch screen, a keyboard, a mouse, a joystick, a projector audio output, a microphone, an image-capture device, a liquid crystal display (LCD) screen and/or a speaker.

The processing circuitry 134 may be configured to execute various operations associated with the system 100. Specifically, the processing circuitry 134 may be configured to execute the one or more operations associated with the system 100 by communicating one or more commands and/or instructions over the communication network 114 to the first and second user devices 102 and 104, the imaging device 108, the plurality of sensors 110, and other entities in the system 100.

The database 202 may be configured to store logic, instructions, circuitry, interfaces, and/or codes of the processing circuitry 200 to enable the processing circuitry 200 to execute the one or more operations associated with the system 102. The database 202 may be further configured to store therein, data associated with the system 102, and the like. It will be apparent to a person having ordinary skill in the art that the database 202 may be configured to store various types of data associated with the system 102, without deviating from the scope of the present disclosure. Examples of the database 202 may include but are not limited to, a Relational database, a NoSQL database, a Cloud database, an Object oriented database, and the like. Further, the database 202 may include associated memories that may include, but is not limited to, a Read-Only Memory (ROM), a Random Access Memory (RAM), a Flash Memory (FM), a Removable Storage Drive (RSD), a Hard Disk Drive (HDD), a Solid State Memory (SSM), a Magnet Storage Drive (MSD), a Programmable Read Only Memory (PROM), an Erasable PROM (EPROM), and/or an Electrically EPROM (EEPROM). Embodiments of the present disclosure are intended to include or otherwise cover any type of the database 202 including known, related art, and/or later developed technologies. In some embodiments of the present disclosure, a set of centralized or distributed network of peripheral memory devices may be interfaced with the information processing apparatus 114, as an example, on a cloud server.

The processing circuitry 134 may be configured to perform one or more operations associated with the system 100 by way of the registration engine 206, the training engine 208, the data collection engine 210, the data processing engine 212, and the display engine 214. In some embodiments of the present disclosure, the registration engine 206 may be configured to enable the first and second users to register into the system 100 by providing registration data through a registration menu (not shown) of the VR application 114 displayed through the first and second user devices 102 and 104, respectively. The registration data may include, but is not limited to, a name, a demographics, a contact number, an address, and the like. Embodiments of the present disclosure are intended to include or otherwise cover any type of the registration data. In some embodiments, the registration engine 206 may be further configured to enable the first and second users to create a login identifier and a password that may enable the first and second users to subsequently login into the system 100. The registration engine 206 may be configured to store the registration data associated with the first and second users, the login and the password associated with the first and second users in a Look Up Table (LUT) (not shown) provided in the database 210.

The training engine 208 may be configured to display a set of predefined training modules in an VR environment that is generated for the first user by way of the VR headset 106. The set of predefined training modules may be displayed when the first user wears the VR headset 106 and activates the training session by way of the first user device 102. For example, the set of predefined training modules may include, but is not limited to, a Salad Module, a Mac N' Cheese Module, a Chicken Strips Module, and the like. Specifically, each module of the set of predefined training modules may be focused on a different element of kitchen safety and achievement. Although, as discussed, the set of predefined training modules includes three training modules (i.e., the Salad Module, the Mac N' Cheese Module, and the Chicken Strips Module), it will be apparent to a person skilled in the art that the scope of the present disclosure is not limited to it. In various other embodiments of the present disclosure, the set of predefined training modules may include more than three modules, without deviating from the scope of the present disclosure. In such a scenario, each module may be focused on a different element of a skill that is to be developed in the first user. Once, the set of predefined modules are displayed in the VR environment of the first user by way of the VR headset 106, the ASD training engine 208 may be configured to generate and provide one or more real-time commands to the first user by way of the VR headset 106 such that in response to the real-time commands, the first user interacts with one or more elements of the VR environment. In other words, the training engine 208 may be configured to generate and provide the one or more real-time commands to the first user inside the VR headset 106 such that the first user is led by a virtual instructor (by way of the one or more real-time commands) to point and make selections throughout a module of the set of predefined modules. In some embodiments of the present disclosure, the real-time commands may be, but not limited to, a visual command (e.g., in textual form that is displayed within the VR environment), a voice over, and the like. Embodiments of the present disclosure are intended to include and/or otherwise cover any type of the commands that can guide the first user to navigate through various options displayed in the VR environment, without deviating from the scope of the present disclosure. In some embodiments of the present disclosure, in response to the commands, the first user may select at least one module of the set of training modules. Specifically, the training engine 208 (in communication with the data processing engine 212) may identify (as discussed below) the at least one module selected by the first user. In some embodiments of the present disclosure, the training engine 208 may be configured to generate a module specific VR environment for the first user based on the selected at least one training module of the set of training modules. Specifically, the module specific VR environment may be generated by way of a pre-recorded live footage that is associated with the selected at least one training module of the set of predefined training modules. For example, when the training engine 208 identifies (based on one or more inputs from the data processing engine 212) that the first user selected the Salad Module, the training engine 208 may generate the module specific VR environment associated with the Salad Module by way of a pre-recorded live footage that is associated with the Salad Module. In some embodiments of the present disclosure, the pre-recorded live footage may include live scenes from a live session that is recorded with real world objects to make the salad by following the stepwise instructions of the Salad Module. Specifically, the pre-recorded live footage utilized for the module specific VR environment may facilitate the first user (i.e., the autistic participants) to practice skills in a safe environment without any real-world consequences to build muscle memory and repetition. Further, the module specific VR environment may facilitate the first user (i.e., the autistic participants) who also quite commonly have other mental health disorders that challenge learning, to be fully focused on a task. Unlike the real world, the module specific VR environment may facilitate in elimination of irrelevant stimuli such that the first user can reduce a learning curve time and gain skills faster. Moreover, autistic adolescents (e.g., the first user) are typically difficult to engage in traditional group therapy sessions. Thus, the module specific VR environment may efficiently engage youth in a deep passion for wanting to come to a behavioral healthcare clinic. In some embodiments of the present disclosure, each training module of the set of training modules may include one or more stepwise instructions to perform one or more tasks. Specifically, each task of the one or more tasks may facilitate to enhance a skill of the first user. For example, the Salad Module may include stepwise instructions to teach the first user (i.e., the user having any type of neurodiverse condition) (i) how to use a knife safely, (ii) the importance of food hygiene by washing the produce, (iii) how to put ingredients together to create a meal, and the like. Similarly, the Mac N' Cheese Module may include stepwise instructions to teach the first user (i.e., the user having ASD) how to safely handle equipment (e.g., boiling pots of water while multi-tasking following step-by-step instructions). Similarly, the Chicken Strips Module may include stepwise instructions to teach the first user (i.e., the user having any type of neurodiverse condition) (i) how to read instructions to correctly set an oven temperature and (ii) how to use a timer to monitor baking progress. In some embodiments of the present disclosure, each task of the one or more tasks may include a plurality of actions to be performed by the first user. Specifically, the plurality of actions may include (i) an action performed in a correct order and (ii) an action performed in an incorrect order. In some embodiments of the present disclosure, the training engine 208 may be configured to generate and provide a real-time command of the one or more commands to the first user to guide the first user. Specifically, in response to the real-time command, the first user may perform at least one action of the plurality of actions. For example, when the real-time command instructs the first user to perform at least one action of the plurality of actions, the first user may perform a movement. The training module engine 208 in communication with the data processing engine 212 may determine the action performed by the first user based on the detected movement performed by the first user (i.e., the detected movement based on the one or more sensed signals received from the plurality of sensors 110). Further, based on the at least one action the plurality of actions performed by the first user, the training module engine 208 may be configured to generate and provide a message to the first user. In some embodiments of the present disclosure, the message may be provided to the first user within the module specific VR environment. The message may be provided by a medium such as, but not limited to, a textual format, an audio format, and the like. Embodiments of the present disclosure are intended to include and/or otherwise cover any type of the medium to provide the message to the first user within the module specific VR environment, without deviating from the scope of the present disclosure. In some embodiments of the present disclosure, the message may include, but is not limited to, an appreciation message when the first user performs the action in the correct order, a warning message when the first user performs the action in the incorrect order, and the like. Embodiments of the present disclosure is intended to include and/or otherwise cover any type of the message that corresponds to the type of action performed by the first user, without deviating from the scope of the present disclosure.

In some embodiments of the present disclosure, the data collection engine 210 may be configured to receive one or more sensed signals from the plurality of sensors 110 and a real-time footage of the first user while the first user executes a module of the plurality of modules. In some embodiments of the present disclosure, the one or more sensed signals may represent one or more parameters associated with the first user. In some embodiments of the present disclosure, the data collection engine 210 may be configured to store the one or more sensed signals and the real-time footage in the database 136 for training the system 100. In some embodiments of the present disclosure, the data collection engine 210 may be configured to provide the one or more sensed signals to the data processing engine 212. In some embodiments of the present disclosure, the data collection engine 210 may be configured to collect data associated with a group of individuals (i.e., similar to the first user that utilizes the system 100 to build skills) and further provide the data to the data processing engine 212. In some embodiments of the present disclosure, the data collection engine 210 may be configured to store the data associated with the group of individuals into the database 136. Further, the data collection engine 210 may be configured to provide the real-time footage to the display engine 214.

The data processing engine 212 may be configured to receive the one or more sensed signals from the data collection engine 210. Further, the data processing engine 212 may be configured to identify an action performed by the first user based on one or more sensed signals received from the plurality of sensors via the data collection engine 210. In some embodiments of the present disclosure, the data processing engine 212 in communication with the training engine 208 may be configured to generate and provide interactive outputs to the first user within the module specific VR environment based on the sensed signals. The data processing engine 212 may facilitate the training engine 208 to identify an action performed by the first user based on the sensed signals received from the plurality of sensors 110. In some embodiments of the present disclosure, the data processing engine 212 may be further configured to process data (i.e., the sensed signals and the real-time footage of the first user). Further, the data processing engine 212 may be configured to process the data associated with the group of individuals (i.e., similar to the first user that utilizes the system 100 to build skills). Specifically, the data processing engine 212 may be configured to describe a distribution of demographic information and one or more clinical characteristics of the group of individuals (i.e., similar to the first user that utilizes the system 100 to build skills) based on descriptive statistics (i.e., a mean and a standard deviation for continuous data and a percentage for categorical data). Further, the data processing engine 212 may be configured to assess a normality through Levene's test of normality for continuous variables. Specifically, the difference in within-group and between-groups comparison will be evaluated with paired sample t-test and independent t-test, respectively for normally distributed variables. In some embodiments of the present disclosure, the data processing engine 212 may be configured to utilize Wilcoxon Rank Sum Test and Mann Whitney U Test for non-normally distributed variables. In some embodiments of the present disclosure, the data processing engine 212 may be configured to compare a difference between categorical variables by way of Chi Square Test and Fisher Exact Test. In some embodiments of the present disclosure, the data processing engine 212 may be configured to determine a success percentage associated with the first user. Specifically, the data processing engine 212 may be configured to, based on the real-time command that instructs the first user to perform at least one action of the plurality of actions, determine the success percentage. For example, when the first user performs x number actions in the correct order out of y possible choices, the data processing engine 212 may determine the success percentage In some embodiments of the present disclosure, when the data processing engine 212 determines that the success percentage is greater than or equal to 80%, the data processing engine 212 may generate a success report. In some embodiments of the present disclosure, each training module of the set of training modules may include one or more one or more questions related to the training module (and/or a therapy program) that the first user is participating in. The data processing engine 212 may be configured to track an answer provided by the first user to each question of the one or more questions. Specifically, the data processing engine 212 may be configured to track a progress of a performance of the first user by collecting data (i.e., the answers to the one or more questions). Further, the data processing engine 212 may generate a score at the end of the training module that represents a number of correct answers out of the total number of questions asked from the first user. For example, when the first user answers 14 out of 20 questions correctly, the data processing engine 212 displays a score of 14/20 to indicate a level of proficiency in the training module being covered.

Specifically, the success report may include a progress of the first user and the generated score of the first user such that when the progress of the first user is greater than or equal to 80%, for 3 months, the first user can practice the skill in real world. In some embodiments of the present disclosure, the data processing engine 212 may be further configured to generate a report that may include information such as, but not limited to, the distribution of demographic information and one or more clinical characteristics of the group of individuals, the assessed normality for the continuous variables, the evaluated difference in within-group and between-groups, and the like. Embodiments of the present disclosure are intended to include and/or otherwise cover any type of the information, without deviating from the scope of the present disclosure. The data processing engine 212 may be configured to provide the generated report to the display engine 214.

The display engine 214 may be configured to receive the real-time footage from the data collection engine 210. Further, the display engine 214 may be configured to generate a display signal that includes the real-time footage. Specifically, the display engine 214 may be configured to generate the display signal in real-time and further transmit the display signal to the second user device 104. In some embodiments of the preset disclosure, the display signal may enable the second user device 104 that may be configured to display the real-time footage of the first user while the first user performs a task of the one or more tasks of the set of predefined modules. Specifically, the real-time footage of the first user displayed by way of the second user device may enable the second user to monitor and track the first user while the first user performs a task of the one or more tasks of the set of predefined modules. In some embodiments of the present disclosure, the second user may be, but is not limited to, a professional (including clinicians who provide services to users with autism and who may adopt practices based on data generated by the system 100), a researcher (who may use the data generated by the system 100 to inform new studies), policy makers (who are essential in effecting change), a family member and/or caretaker of the first user (i.e., a person having autism), and the like. The display engine 214 may be further configured to generate a report signal that may include the generated report. Further, the display engine 214 may be configured to transmit the report signal to the second user device 104. The report signal may enable the second user device 104 to display the report embedded in the report signal by way of the second interface 126.

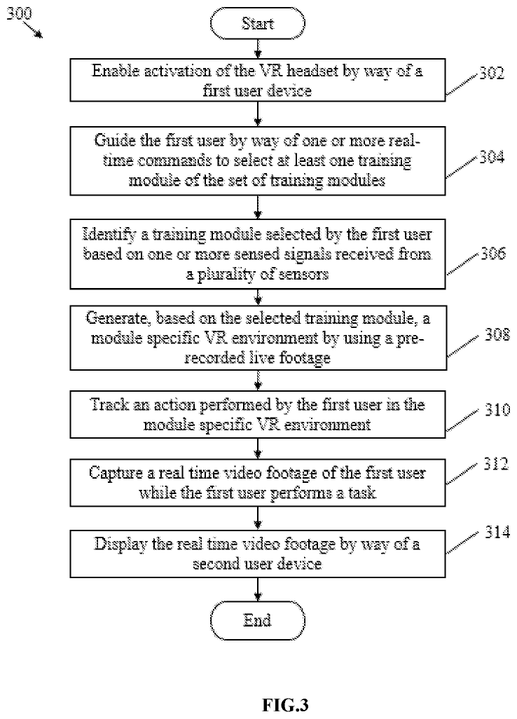

FIG. 3 illustrates a flowchart of a method 400 for training the first user (i.e., an individual with ASD) by way of the Virtual Reality (VR), in accordance with an embodiment of the present disclosure.

At step 302, the system 100 enables the first user to activate the VR headset 106 worn by the first user by way of the first user device 102 such that upon activation, the VR headset 106 displays a set of predefined training modules within a VR environment.

At step 304, the system 100 may guide the first user by way of one or more real-time commands to select at least one training module of the set of training modules. In some embodiments of the present disclosure, each training module of the set of training modules may include one or more stepwise instructions to perform one or more tasks such that each task is adapted to enhance a skill of the first user. Further, each task of the one or more tasks may include a plurality of actions to be performed by the user, wherein the plurality of actions may include (i) an action performed in a correct order and (ii) an action performed in an incorrect order.

At step 306, the system 100 may sense by way of the plurality of sensors 100, one or more signals representing parameters associated with the first user. Specifically, the system 100 may identify at least one of the plurality of actions performed by the first user based on one or more sensed signals received from the plurality of sensors. In some embodiments of the present disclosure, based on the guidance and the one or more sensed signals, the system 100 may identify the training module selected by the first user.

At step 310, the system 100 may generate, based on the selected at least one training module of the set of training modules, a module specific VR environment for the first user such that the module specific VR environment is generated by way of a pre-recorded live footage that is associated with the selected at least one training module of the set of training modules.

At step 312, the system 100 may guide the first user by way of one or more real-time commands to perform a task of the one or more tasks. Further, the system 100 may identify and track an action of the plurality of actions performed by the first user based on one or more sensed signals received from the plurality of sensors while the first user performs the task of the one or more tasks. The system 100 may further generate a score by tracking an answer provided by the first user to each question of one or more questions associated with the selected at least one training module of the set of training modules.

At step 314, the system 100 may capture, by way of one or more imaging devices, a real time video footage of the first user while the first user performs the task of the one or more tasks.

At step 316, the system 100 may display, by way of the second user device associated with the second user, the real time video footage such that the second user can monitor the first user while in the module specific VR environment.

Thus, the system 100, the information processing apparatus 112, and the method 300 provide a fast, safe, and effective learning platform that engages an individual with ASD (e.g., the first user) in therapeutic interventions and evidence-based behavioral health treatment services. Further, the system 100, the information processing apparatus 112, and the method 300 can improve psychosocial functioning and quality of life by raising the individual's level of independence in activities of daily living. The system 100, the information processing apparatus 112, and the method 300 facilitate to provide lower healthcare costs for the autistic population through innovative and preventative healthcare by reducing the ongoing service need into adulthood.

While various embodiments of the present disclosure have been illustrated and described, it will be clear that the present disclosure is not limited to these embodiments only. Numerous modifications, changes, variations, substitutions, and equivalents will be apparent to those skilled in the art, without departing from the spirit and scope of the present disclosure, as described in the claims. Further, unless stated otherwise, terms such as “first” and “second” are used to arbitrarily distinguish between the elements such terms describe. Thus, these terms are not necessarily intended to indicate temporal or other prioritization of such elements.

1. An information processing apparatus comprising:
processing circuitry that is coupled to a Virtual Reality (VR) headset that is adapted to be worn by a first user, wherein the processing circuitry is configured to:
display, by way of the VR headset, a set of predefined training modules within a VR environment such that upon display of the set of training modules, the first user selects at least one training module of the set of training modules; and
generate, based on the selected at least one training module of the set of training modules, a module specific VR environment for the first user such that the module specific VR environment is generated by way of a pre-recorded live footage that is associated with the selected at least one training module of the set of training modules.

2. The information processing apparatus of claim 1, wherein each training module of the set of training modules comprising one or more stepwise instructions to perform one or more tasks such that each task is adapted to enhance a skill of the first user.

3. The information processing apparatus of claim 2, wherein each task of the one or more tasks comprising a plurality of actions to be performed by the user, wherein the plurality of actions comprising (i) an action performed in a correct order and (ii) an action performed in an incorrect order.

4. The information processing apparatus of claim 3, wherein the processing circuitry is further configured to identify one of the plurality of actions performed by the user based on one or more sensed signals received from a plurality of sensors, wherein the one or more sensed signals represents one or more parameters associated with the first user.

5. The information processing apparatus of claim 1, wherein the processing circuitry is further configured to:
capture, by way of one or more imaging devices, a real time video footage of the first user while the first user performs a task of the one or more tasks; and
display, by way of a second user device associated with a second user, the real time video footage.

6. The information processing apparatus of claim 5, wherein the processing circuitry is further configured to enable the second user, by way of the second user device, to (i) provide one or more instructions to the first user, (ii) monitor the first user, or a combination thereof.

7. The information processing apparatus of claim 5, wherein the processing circuitry is further configured to enable, by way of the second user device, the second user to provide real-time commands to the first user within the module specific VR environment.

8. The information processing apparatus of claim 1, wherein the processing circuitry is further configured to generate a score, wherein to generate the score, the processing circuitry is configured to track an answer provided by the first user to each question of one or more questions associated with the selected at least one training module of the set of training modules.

9. A system comprising:
a Virtual Reality (VR) headset that is adapted to be worn by a first user;
processing circuitry that is coupled to the VR headset, and configured to:
display, by way of the VR headset, a set of predefined training modules within a VR environment such that upon display of the set of training modules, the first user selects at least one training module of the set of training modules; and
generate, based on the selected at least one training module of the set of training modules, a module specific VR environment for the first user such that the module specific VR environment is generated by way of a pre-recorded live footage that is associated with the selected at least one training module of the set of training modules.

10. The system of claim 9, wherein each training module of the set of training modules comprising one or more stepwise instructions to perform one or more tasks such that each task is adapted to enhance a skill of the first user.

11. The system of claim 10, wherein each task of the one or more tasks comprising a plurality of actions to be performed by the user, wherein the plurality of actions comprising (i) an action performed in a correct order and (ii) an action performed in an incorrect order.

12. The system of claim 9, further comprising a plurality of sensors configured to sense signals that represent one or more parameters associated with the user, wherein, the processing circuitry identifies one of the plurality of actions performed by the user based on the one or more sensed signals.

13. The system of claim 9, further comprising a timer coupled to the processing circuitry such that the processing circuitry activates the timer when the first user selects at least one training module of the set of training modules.

14. The system of claim 9, further comprising a second user device that is configured to enable a second user to (i) provide one or more instructions to the first user, (ii) monitor the first user, or a combination thereof.

15. The system of claim 9, further comprising one or more imaging devices that are coupled to the processing circuitry, and configured to capture a real time video footage of the first user while the first user performs each task of the one or more tasks, wherein the processing circuitry is further configured to display the real time video footage by way of the user device associated with a second user.

16. The system of claim 9, wherein the processing circuitry is further configured to generate a score, wherein to generate the score, the processing circuitry is configured to track an answer provided by the first user to each question of one or more questions associated with the selected at least one training module of the set of training modules.

17. A computer-implemented method that when executed on processing circuitry causes the processing circuitry to perform operations comprising:
displaying, by way of a VR headset, a set of predefined training modules within a VR environment such that upon display of the set of training modules, a first user selects at least one training module of the set of training modules; and
generating, based on the selected at least one training module of the set of training modules, a module specific VR environment for the first user such that the module specific VR environment is generated by way of a pre-recorded live footage that is associated with the selected at least one training module of the set of training modules.

18. The method of claim 17, wherein each training module of the set of training modules comprising one or more stepwise instructions to perform one or more tasks such that each task is adapted to enhance a skill of the first user.

19. The method of claim 18, wherein each task of the one or more tasks comprising a plurality of actions to be performed by the user, wherein the plurality of actions comprising (i) an action performed in a correct order and (ii) an action performed in an incorrect order.

20. The method of claim 17, further comprising sensing by way of a plurality of sensors, one or more signals representing parameters associated with the first user.

21. The method of claim 17, further comprising identifying, by way of the processing circuitry, at least one of the plurality of actions performed by the first user based on one or more sensed signals received from the plurality of sensors.

22. The method of claim 17, further comprising:
capturing, by way of one or more imaging devices, a real time video footage of the first user while the first user performs each task of the one or more tasks; and
displaying, by way of the second user device, the real time video footage.

23. The method of claim 17, further comprising generating, by way of the processing circuitry, a score by tracking an answer provided by the first user to each question of one or more questions associated with the selected at least one training module of the set of training modules.
